(19) United States
(12) Patent Application Publication
(10) Pub. No.: US 2015/0238514 A1
(43) Pub. Date: Aug. 27, 2015

(54) Protective Supplement Configuration

(71) Applicant: William Armstrong, Carlsbad, CA (US)

(72) Inventor: William Armstrong, Carlsbad, CA (US)

(21) Appl. No.: 14/187,134

(22) Filed: Feb. 21, 2014

(51) Int. Cl. A61K 31/7084; A61K 31/122; A61K 9/50

(52) U.S. Cl. CPC A61K 31/7084; A61K 9/5073; A61K 9/5015; A61K 31/122

(57) ABSTRACT

An orally ingested supplement is provided having a core formed of NADH which is protected from communication with stomach acid once ingested by both an enteric coating on the core and a surrounding secondary layer formed of CoQ10 which acts to increase energy output from the NADH. Other supplements may be included in the secondary layer and a plurality of cores may also be included with each surrounded by a protective secondary layer.

### BACKGROUND OF THE INVENTION

1. Field of the Invention

The present invention relates to supplements. More particularly it relates to supplements which may be provided in combination which have a symbiotic relationship in nutrition, and a configuration and method of insuring ingested combinations are absorbed and have sufficient quantity of each ingredient to provide a maximized nutritional supplement to users.

2. Prior Art

It is well known that cellular energy production in animals and humans requires both NADH and Coenzyme Q10 and good health and energy and in humans clear thinking requires an adequate supply of both supplements. Both NADH and Coenzyme Q10 are found in foods, however in a modern world with many types of food processing, pasteurization, nuclear pasteurization, and high pressure pasteurization techniques, many nutritional elements present in food are destroyed, or impaired by the processing.

It is well known that there is a symbiotic relationship between NADH and Coenzyme Q10 in cellular energy production in humans and animals. In this relational process, Coenzyme Q10 is employed during cellular energy production however this involvement occurs at a much lower level than NADH.

In this cellular process, Coenzyme Q10 requires an adequate available supply of NADH to reduce the Coenzyme Q10 to its reduced form in which it is sufficiently active and can transport electrons from NADH to oxygen molecules which directly leads to cellular energy production in form of ATP.

Consequently, in order for CoQ10 to perform its function in this body energy production scheme, a sufficient supply of NADH must be maintained and also be readily available. However, as noted, food processing, cooking, and poor soil conditions for food growth and other issues can all lead to an inadequate supply of NADH in consumable food. Further, while cellular energy production can occur with NADH without CoQ10 its presence, energy production in a symbiotic relationship with CoQ10 as a catalyst, significantly adds to the energy producing capacity of NADH when taken in conjunction.

Still further, many studies show that NADH has additional functions in the body in addition of producing energy. NADH is known to help repair cell and DNA damage and additionally to stimulate the immune system. Further NADH is reported to stimulate the biosynthesis of the neurotransmitters, adrenaline, dopamine and serotonin. Additionally, NADH is known to stimulate the production of NO (Nitric Oxide) which induces relaxation of blood vessels in many organs such as heart, lung, brain and kidney.

As can be discerned, an adequate supply of NADH in humans and animals is a requirement for good mental and physical health. Further, while an adequate supply of NADH is desirable at all times, to significantly increase energy readily available from NADH, an adequate supply of CoQ10 is also required.

In addition to NADH and CoQ10 for an energy combination, there are many other combinations of supplements which can benefit users when taken in combination and concurrently provide a protective shell to NADH. For example to help users with arthritis=NADH in a protective surrounding formed of one or a combination of ingredients including Glucosamine, Chondroitin sulphate, MSM. Also, for eyesight, NADH with a surrounding shell of L-arginine. These two examples of course are but a pair of many, and should not be limiting. Using the additional supplements to surround and protect the NADH core with any such additional individual or plurality of supplements is considered within the scope of this invention.

Unfortunately both of these nutritional elements as noted, are not always present in food in sufficient quantities. Of additional concern in nutrition is the fact that NADH can easily be destroyed by stomach acid. As such, supplementation of both NADH and CoQ10 where both are readily available at the same time, to cells for energy production, has been a challenge in the current art.

Because of its easy destruction by stomach acid, NADH supplementation in the current art has been achieved through intravenous means or using a sublingual delivery in order to protect this easily destroyed supplement from exposure to stomach acid. For people in need of NADH supplementation, the lack of a means to orally supplement their diet significantly limits their ability to achieve sufficient levels in their body since sublingual modes are not widely available and limited in delivery amount, and since intravenous delivery is not an attractive option to most people.

As such, there is a continuing unmet need for a means for oral supplementation of NADH which will deliver all or a majority of the NADH supplement ingested, into the small intestine undamaged. Such a delivery mode should be in a tablet or capsulated supplement formulation which has sufficient protection from exposure to stomach acid to allow sufficient time for orally ingested supplements to arrive in the intestines unscathed.

Further, due to the significant gain in energy production achieved from the catalyst effect of CoQ10, to maximize the energy provided from NADH, one mode of such a supplement should be provided in combination with CoQ10 as the protectant to insure its presence concurrently, during cellular energy production. In such a combination supplement formulation of both CoQ10 and NADH, the supplement in order to make it easy to employ and widely available should be provided in an orally ingested tablet. This tablet or capsule should include means for protection of the supplement from exposure to stomach acids, for a sufficient time to pass to the small intestine, whereby a concurrent delivery and thus availability of both supplements passes to the intestines undamaged to insure maximized cellular energy production.

Still further, other combinations of NADH with other supplements which can benefit users when taken, should be provided in combination with NADH for energy. Such combinations should form one or more protective shells around a center-positioned supply of NADH to insure delivery.

The forgoing examples of prior art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The device herein disclosed and described provides a solution to the shortcomings in current art in the provision in one preferred mode, of a supplement tablet or capsule of NADH which is surrounded by one or a plurality of protective shells formed of other nutritional supplements and or enteric coating layers. In another mode, the disclosed supplement provides a combination of NADH dehydrogenase (ubiquinone, and CoQ10 to insure the availability of an adequate and timely supply of CoQ10 to symbiotically interact to maximize the potential for the catalyst effect on the energy for cells derived from employment of NADH.

The supplement of NADH with other supplements surrounding it, or formed in a central core of NADH with an hemispheric layer of CoQ10 surrounding the NADH, in a combination of supplements is provided in a tablet or capsule form. Enteric coatings may be added for protection of the supplements, and/or to insure the timing of uptake by the user's body such that there is an adequate supply of both supplements in their body in a timely fashion for combination in the symbiotic action which dramatically increases the energy derived. The tablet or capsule formed in the various modes herein disclosed, may be orally ingested because it includes one or a plurality of layers surrounding the NADH and/or enteric coatings both of which singularly or in combination protect the central core of NADH from stomach acid for sufficient time to survive passage through the acid exposure in the stomach, and enter the small intestine for absorption.

Survival of the capsule or tablet bearing NADH or the combination of NADH AND CoQ10 or other supplements, is accomplished by positioning one or a combination of a hemispheric layer of CoQ10 surrounding the NADH and/or an enteric coating of an average enteric coating thickness is 0.123″ with thickness specifications from tablet compression 0.080″-0.140.″ If employed in a preferred mode of the device and method herein, the coating is positioned on the exterior of the formed core of solid NADH or formed of a plurality of micro tablets formed of enteric coated NADH which is then surrounded with an exterior layer of CoQ10 which may be surrounded by gelatin or a edible polymeric or other coating or an enteric coating layer.

Experimentation showed results yielding a number of problems with the timing of absorption by the body, of both supplements, in a fashion to insure a presence of CoQ10 for use by cells in a combination with the NADH. In some configurations the enteric coating was too thick and passed through the intestines unabsorbed. In some configurations tested, the core of NADH was absorbed by the small intestine, but prior to absorbing of the majority of CoQ10.

After numerous attempts using different coating ingredients and thicknesses, a timing of absorption of both components was accomplished wherein CoQ10 was absorbed by the user's body in sufficient quantity, and at a time early enough, to be present in the body and system of the user when the NADH is absorbed by the small intestine. This timing was achieved by placing seaweed based enteric coating in a range of between 0.9 miles and 1.4 mils with a particularly good results using a thickness of 1.23 mils, on the exterior surface of the core component formed of the supplement of NADH. Enteric coating layers outside the range noted yielded less than satisfactory timing results.

The enteric coated core of NADH in the formed tablet or capsule, has a hemispheric layer of CoQ10 surrounding the NADH core. The CoQ10 itself is surrounded by gelatin or an edible polymer or a similar protective non enteric coating.

In this configuration, when the tablet or capsule is swallowed, the CoQ10 and absorbed by the body first, while concurrently providing means for protecting the NADH from contact with stomach acid, and is thus present in the body when the enteric coating surrounding the NADH dissipates in the small intestine allowing absorption of the NADH, and will replenish the existing body supply of NADH used in combination with the CoQ10.

The NADH used is immediately replenished within a two hour to three hour time frame, when the coating on the NADH portion of the tablet or capsule is dissolved and small intestine absorbs it. After experimentation yielded predicted outcomes wherein the timing of absorption was not ideal to provide absorption and the replenishment of the NADH for use in combination with CoQ10 in sufficient quantities for an ideal reaction and energy production increase, success was shown when a coating of seaweed based enteric coatings of the thickness showed it would in a timed fashion, survive and be absorbed by the small intestine in a two hour window.

While this two hour timing and configuration of the supplement table with the noted interior enteric coating worked exceptionally well, the availability of the NADH to the small intestine it was found could be in a shorter timing using a thinner coating, or longer timing if a thicker enteric coating surrounds the NADH.

However, after much experimentation, it was noted that an average time for the NADH to pass through a user's intestinal tract is 1.5 to 2.5 hours, and that the NADH must be available and absorbable in that time frame to replenish body supplies or it would be exiting to the large intestine and be subsequently expelled. Ideally, it should be available during the portion of time between 0.5 and 2 hours after passage from the stomach, to allow a reasonable amount of time for absorption subsequent to passage through stomach acid, and before being expelled to the large intestine, and to allow some margin of time for differing digestive tracts of different users. Thus the thickness of the enteric coating, and the type, should be configured to allow passage through the stomach acid safely to the small intestine, and thereafter sufficiently be dissolved, within 30 minutes of passage into the small intestine to provide this window of 0.5 to 2.0 hours for absorption and resupply the body for NADH used.

Various configurations of NADH and combinations with CoQ10 were tested in a simulated stomach environment using SGF (simulated gastric fluid) and SIF (simulated intestinal fluids). Through numerous bouts of such testing it was found that when an enteric coated NADH tablet or capsule, is swallowed and digested with a glass of water on an empty stomach, the coating will delude the stomach into discerning just water has been ingested and in a subsequent reaction, eject the water and tablet from the stomach into the small intestine where the enteric coating can dissolve and the NADH can be absorbed before being expelled.

With such information discerned after much experimentation, a mode of the device herein was arrived upon wherein an enteric coating film or layer, is applied to the stabilized NADH tablet forming a core, or a plurality of individual micro tablets forming a core, in an enteric coating thickness and of a material which if not passed immediately to the small intestine, will outlast the stomach acid for a period of 2 hours and 45 minutes.

This mode of the invention has been found to insure that the tablet of NADH, in an absorbable fashion is positioned within the small intestine at a point, and for a duration, where it may be fully absorbed. This was tested in SGF (simulated gastric fluid) and SIF (simulated intestinal fluids).

The NADH as noted may be surrounded by CoQ10 for placing both supplements in supply for optimum energy production, and/or the NADH can be surrounded by other supplements. Further, while a seaweed-based enteric coating has shown to work exceptionally well in the preferred mode, a pharmaceutical type enteric coating may be employed so long as it is sufficient to survive for a period from 2 hours to 3 hours, and optimally for 2 hours and 45 minutes.

Further, in another preferred mode of the device shown to work well in experimentation, the NADH is formed of a plurality enteric coated micro-tablets which are formed in a solid solution of NADH. This mode insures that at least some of the NADH gets past the stomach acid, if for some reason an enteric coating fails due to something the user ingested causing a reaction, or if a coating layer is too thin or eroded, or of some of the micro-tablets are in the stomach too long for some other reason.

In another favored mode of the supplement herein, one or a combination of additional supplements, from a group of additional supplements, may be combined with the stabilized NADH tablet or micro tablets for additional nutrition. This group include Glucosamine, Chondroitin sulphate, Methylsulfonymethane (MSM) blend, and CoQ10.

With respect to the above description, before explaining at least one preferred embodiment of the herein disclosed nutritional supplement invention in detail, it is to be understood that the invention is not limited in its application to the details of construction and to the arrangement of the components in the following description or illustrated in the drawings. The invention herein described is capable of other embodiments and formulations and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other timed nutritional supplements, methods and systems for carrying out the several purposes of the present disclosed supplement. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of,” Thus, the phrase “consisting of” indicates that the listed elements are required or mandatory, and that no other elements may be present. By “consisting essentially of’ is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It is an object of this invention to provide maximized delivery of NADH to the small intestine of users to maximize absorption thereof.

It is an additional object of this invention to provide CoQ10 in a timed relationship with the NADH to maximize energy production from the NADH.

These and other objects of the invention will be brought out in the following part of the specification, wherein detailed description is for the purpose of fully disclosing the invention without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features. It is intended that the embodiments and figures disclosed herein are to be considered illustrative rather than limiting. In the drawings:

FIG. 1 depicts a mode of the supplement in a layered tablet.

FIG. 2 shows a mode of the supplement table wherein a plurality of micro tablets occupies a core.

FIG. 3 shows a view of another mode of the nutritional tablet having a plurality of layered tablets surrounded by a capsule.

FIG. 4 depicts the device of FIG. 3 having cores formed of a plurality of individual sections of NADH.

### DETAILED DESCRIPTION OF THE PREFERRED EMBODIMENTS OF THE INVENTION

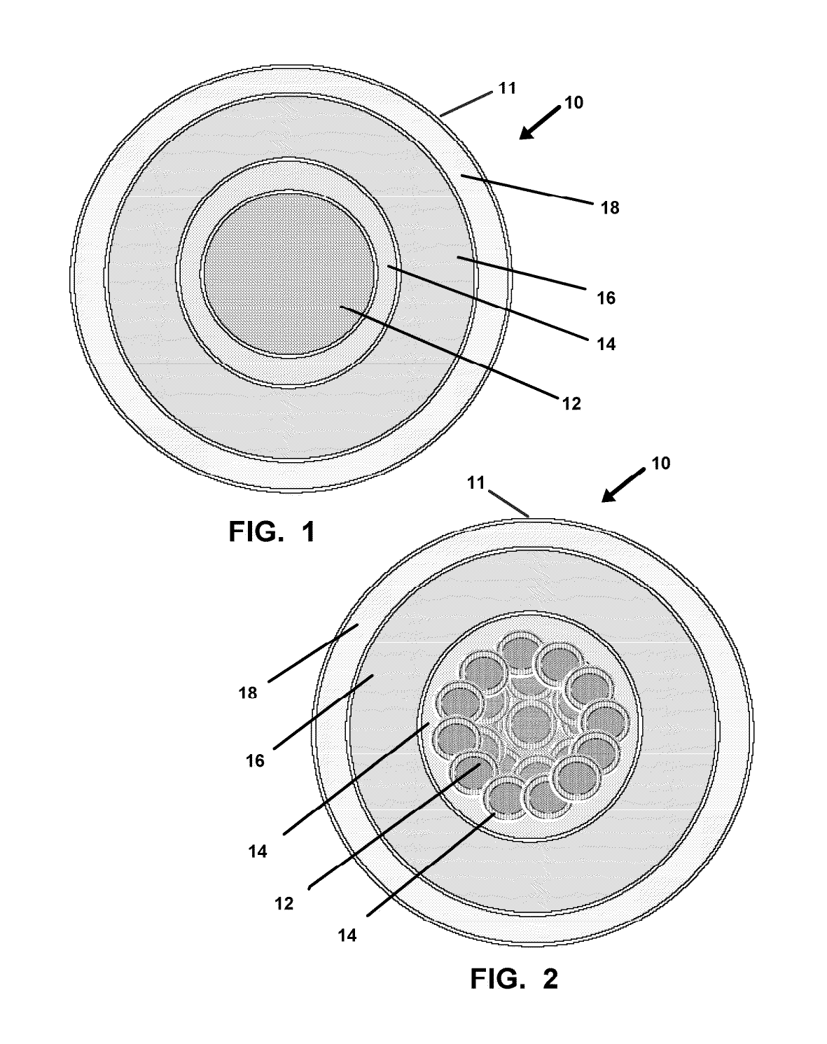

Now referring to drawings in FIGS. 1-3, which depicts some preferred modes of the supplement device 10 and method herein, and wherein similar components are identified by like reference numerals.

There is seen in FIG. 1, a particularly preferred mode of the supplement device 10 shown having a plurality of hemispheric layers or sections forming a tablet 11 or capsule and while the term “tablet” is being used, it could also be a capsule or other mode of ingesting supplements. In a core section, is positioned a portion of NADH 12 which is surrounded by an enteric coating 14 of sufficient thickness, and of an edible enteric material configured to survive between 2 and 3 hours of exposure to stomach acid of humans.

Currently a particularly preferred enteric coating derived from seaweed and therefor natural, is of substantially a thickness of 1.23″ and formed of Sodium Alginate which is particularly preferred. By substantially herein is meant anywhere from 30 percent thinner or 30 percent thicker, however, the enteric coating may be formed of any one or a combination of nutraceutical and pharmaceutical enteric coatings ingredients configured to allow the core of NADH 12 to pass through the stomach acid after substantially two hours of exposure thereto. Substantially being between 25 percent less or more time of exposure. Currently, such enteric coating ingredients may be any one or combination of ingredients from a group including Methacrylic Acid Copolymer, Nutrateric manufactured by Colorcon, Spectrablend sold by Sensient, Acryl-EZE sold by Colorcon, Opadry from Colorcon, Sureteric from Colorcon, and Sodium Alginate or a similar seaweed-based coating.

If provided singularly without a surrounding supply of CoQ10, the core stabilized NADH 12 may be provided in tablet form with the enteric coating 14 forming a first protective layer of a hemispheric covering. However, in a particularly preferred mode of the supplement device 10, which yields maximized energy production from the NADH or the user due to resupply of NADH used with the CoQ10, a secondary hemispheric surrounding layer of CoQ10 is positioned to entirely surround and encapsulate the core-surrounding enteric coating 14 covering the centrally located component formed of NADH 12.

In one preferred mode of the device 10 and method, the core of NADH 12 with enteric 14 coating, is surrounded by a secondary layer 16. In a preferred mode for energy optimization, the secondary layer is formed of CoQ10. So configured the secondary layer 16 may have an outer shell 18 layer or hemispheric surface layer of gelatin or a similar atmosphere-protective but also a non enteric coating.

Alternatively, in all modes of the supplement device 10 herein, other nutritional supplements may be positioned in the core layer in combination with NADH 12, or more preferably, included to form the secondary layer 16, acting as the supplemental protective covering or barrier to stomach acid communicating with the NADH 12. Such supplements may be any one or combination of individual supplements from a group of supplements which may be combined in a surrounding hemispheric layer with the centrally located NADH 12 core formed in tablet or micro tablet form, and formed of one or a combination of supplements in a group including Glucosamine, Chondroitin sulphate, Methylsulfonymethane (MSM) blend, and CoQ10.

In one mode of the device 10 the secondary layer 16 is formed of an one or combination of ingredients from a group including Glucosamine, Chondroitin sulphate and Methylsulfonymethane (MSM), as an arthritis aiding product. It can include the CoQ10 also in the preferred mode, although such might be taken separately if the device 10 is ingested for energy and arthritis and the supplements provide the secondary layer 16 to help protect communication of stomach acid with the core formed of NADH 12.

In another mode of the device 10 the secondary layer 16 may include L-arginine as a supplement for eye macular degeneration, ocular hypertension and glaucoma, in combination with the CoQ10 in the preferred configuration, although such could be taken separately if required while the secondary layer 16 still provides the barrier for stomach acid.

In all modes of the device 10 and method of forming such herein, the secondary layer 16 surrounds the enteric coated core and may be formed of a supplement different from the core of NADH 12 and can be any one, or a combination of supplements from a group Glucosamine, Chondroitin sulphate, Methylsulfonymethane (MSM) L-arginine, and CoQ10. The secondary layer 16 in all modes of the device 10 is formed in a hemispheric covering surrounding the core of NADH 12 to provide the secondary means for enteric protection of the NADH from exposure to stomach acid while the enteric coating 14 around the core of NADH 12 would provide a primary barrier to prevent communication of stomach acid with the NADH 12 core.

FIG. 2 depicts another favored mode of the supplement device 10 wherein the NADH 12 core is formed of micro caplets each having a core of NADH 12 surrounded by a covering of an enteric coating 14. A secondary layer 16 formed of CoQ10 alone, or CoQ10 in a combination of a stabilized supplement, including Glucosamine, Chondroitin sulphate, Methylsulfonymethane (MSM) blend, and the CoQ10, is formed to entirely surrounds the core of NADH 12 formed by the micro caplets which themselves may have an enteric coating, in combination with an enteric coating 14 surrounding the micro caplets of NADH 12 having individual enteric coatings 14 thereon.

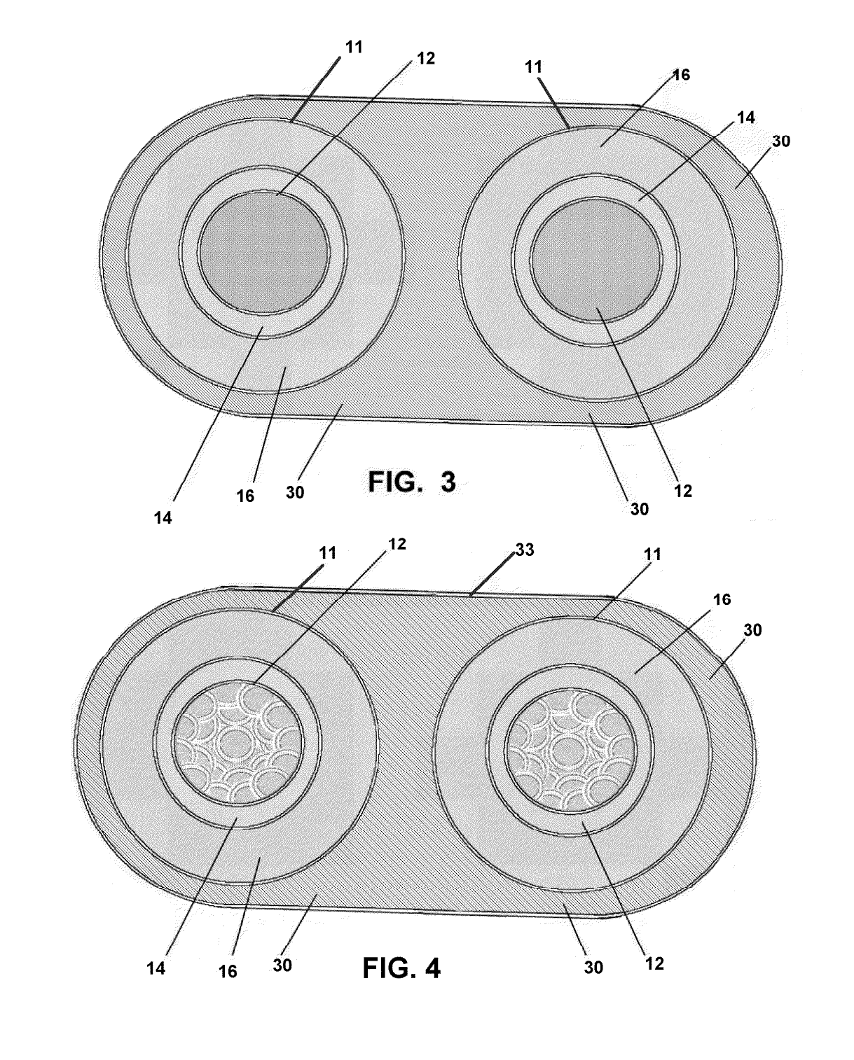

FIG. 3 depicts another mode of the device 10 having a pair of supplement tablets or capsules in the mode of the device 10 of FIG. 1. FIG. 4 shows another mode of the device 10 having a pair of supplements formed in the manner of the device of FIG. 2, although, either of the device 10 shown in FIG. 1 or FIG. 2, or combinations thereof may be employed.

In FIGS. 3-4 also shown is a third layer 30 surrounding the pair of tablets 11, which may be gelatin or an inert but edible material, or may be formed as a third layer 30 of other nutritional supplements as would occur to those skilled in the art, in a singular or combination mixture thereof, or could be air. This third layer 30 offers a third means to protect communication with the core of NADH 12 by stomach acid and a supplemental means for inclusion of supplements in combination with those in tablets 11 of the invention.

It is additionally noted and anticipated that although the device is shown in its most simple form, various configurations and aspects of the device may be differently shaped or slightly modified when forming the invention herein. As such those skilled in the art will appreciate the descriptions and depictions set forth in this disclosure or merely meant to portray examples of preferred modes of the invention within the overall scope and intent of the invention, and are not to be considered limiting in any manner.

While all of the fundamental characteristics and features of the supplement and method herein have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed:

1. An orally ingested supplement, comprising:
a first core defined by a first core surface, said first core formed of NADH;
an enteric coating positioned on said first core surface;
a secondary layer totally surrounding said first core surface;
said secondary layer formed of a second nutritional supplement other than said NADH;
said enteric coating forming a first barrier providing a first means for prevention of communication of stomach acid with said first core subsequent to an ingestion by a user of said;
said secondary layer forming a second barrier providing a second means for prevention of communication of said stomach acid with said first core; and
whereby a combination of said first barrier and said second barrier is configured to maintain said prevention of said communication of said stomach acid with NADH of said first core, for a period sufficient to pass from said user's stomach to said small intestine.

2. The orally ingested supplement of claim 1, additionally comprising:
said secondary layer formed of CoQ10 and having a first surface surrounding said first core surface;
said secondary layer having a covering layer surrounding a second surface thereof positioned opposite said first surface; and
said covering providing a barrier to a communication of said CoQ10 with a surrounding atmosphere.

3. The orally ingested supplement, of claim 1, additionally comprising:
said first core defined by said first core surface being formed of a plurality of individual sections of said NADH, each said section having a section surface having a section enteric coating thereon;
each respective said section enteric coating on each said section surface forming a respective third barrier to communication of stomach acid with said NADH of each said respective individual sections covered by said section enteric coating; and
formation of said first core of said plurality of said individual sections, providing means for insuring at least a portion of said NADH contained in said first core, is communicated to said small intestine without having been in a communication with said stomach acid.

4. The orally ingested supplement, of claim 2, additionally comprising:
said supplement having a second core defined by a second core surface, said second core formed of NADH;
an enteric coating positioned on said second core surface;
a secondary layer formed of said CoQ10 totally surrounding said second core surface;
said enteric coating on said second core surface forming a fourth barrier providing a first means for prevention of communication of stomach acid with said NADH in said second core subsequent to an ingestion by a user of said;
said secondary layer totally surrounding said second core forming a fifth barrier providing a second means for prevention of communication of said stomach acid with said NADH in second core;
said covering defining a hemispheric surface surrounding both respective said secondary layers surrounding said first core and said second core; and
whereby said fourth barrier in combination with said fifth barrier are configured to maintain said prevention of said communication of said stomach acid with said second core, once ingested, for said period sufficient to pass from said user's stomach, to said small intestine.

5. The orally ingested supplement of claim 4, additionally comprising:
said first core defined by a said first core surface being formed of a first plurality of individual sections of said NADH, each said section having a section surface having an section enteric coating thereon;
said second core defined by a said second core surface being formed of a second plurality of individual sections of said NADH, each said section having a section surface having an section enteric coating thereon; and
each respective said section enteric coating on each said section respectively forming said first core and said second core, forming a barrier layer preventing communication of stomach acid with said NADH of each said first core and said second core.

6. The orally ingested supplement of claim 1, additionally comprising:
said second nutritional supplement of said second layer formed of one or a plurality of supplement ingredients from a group of supplement ingredients including Glucosamine, Chondroitin sulphate, Methylsulfonymethane (MSM), and CoQ10.

7. The orally ingested supplement of claim 3, additionally comprising:
said second nutritional supplement of said second layer formed of one or a plurality of supplement ingredients from a group of supplement ingredients including Glucosamine, Chondroitin sulphate, Methylsulfonymethane (MSM) blend, and CoQ10.

8. The orally ingested supplement of claim 1, additionally comprising:
said second nutritional supplement of said second layer formed of one or a plurality of supplement ingredients from a group of supplement ingredients including L-arginine and CoQ10.

9. The orally ingested supplement of claim 3, additionally comprising:
said second nutritional supplement of said second layer formed of one or a plurality of supplement ingredients from a group of supplement ingredients including L-arginine and CoQ10.

10. The orally ingested supplement of claim 4, additionally comprising:
said second nutritional supplement of said secondary layer surrounding said first core and said second core formed of one or a plurality of supplement ingredients from a group of supplement ingredients including Glucosamine, Chondroitin sulphate, Methylsulfonymethane (MSM), and CoQ10.

11. The orally ingested supplement of claim 5, additionally comprising:
said second nutritional supplement of said secondary layer surrounding said first core and said second core formed of one or a plurality of supplement ingredients from a group of supplement ingredients including Glucosamine, Chondroitin sulphate, Methylsulfonymethane (MSM), and CoQ10.

12. The orally ingested supplement of claim 4, additionally comprising:
said second nutritional supplement of said secondary layer surrounding said first core and said second core being formed of one or a plurality of supplement ingredients from a group of supplement ingredients including L-arginine and CoQ10.

13. The orally ingested supplement of claim 5, additionally comprising:
said second nutritional supplement of said secondary layer surrounding said first core and said second core being formed of one or a plurality of supplement ingredients from a group of supplement ingredients including L-arginine and CoQ10.
